(19) United States
(12) Patent Application Publication
(10) Pub. No.: US 2011/0152736 A1
(43) Pub. Date: Jun. 23, 2011

(54) Brace having a Force Indicator

(75) Inventor: Tracy Ng, San Francisco, CA (US)

(73) Assignee: DJO, LLC, Vista, CA (US)

(21) Appl. No.: 12/783,434

(22) Filed: May 19, 2010

Related U.S. Application Data
(60) Provisional application No. 61/179,658, filed on May 19, 2009.

(51) Int. Cl. A61F 5/00

(52) U.S. Cl. 602/16

(57) ABSTRACT

A hinge brace includes a hinge unit having medial and lateral plates enclosing a hinge mechanism, a sensing unit operatively connected to the hinge mechanism for sensing a force applied by the brace onto a user's knee. The hinge brace also includes a display unit that is operatively connected to the sensing unit for indicating a level of force applied to the user's knee by the brace.

### CROSS-REFERENCE TO RELATED APPLICATIONS

This application claims the benefit of U.S. Provisional Application Ser. No. 61/179,658, filed May 19, 2009, the entire contents of which are incorporated herein by reference.

### BACKGROUND

Osteoarthritis is a degenerative joint disease that results in chronic pain in an affected joint when the joint is statically or dynamically loaded. In an affected knee joint, osteoarthritis pain is often caused by an unbalanced loading on the medial or lateral compartment of the knee, which can reduce the clearance space between the condyles of the femur and tibial plateau. When there is increased pressure between the femoral and tibial surfaces in an affected compartment of the knee joint, and particularly dynamic pressure, cartilage degeneration can occur at the contact surface producing pain in the joint.

Orthopedic knee braces are commonly applied to the leg to treat osteoarthritis in the knee. Other conditions affecting the knee also use braces for therapy and rehabilitation with an eye to reducing pain and improving post surgical recovery. Such braces typically include an upper support portion for securing to the upper leg of the wearer, a lower support portion for securing to the lower leg, and one or more hinge assemblies pivotally interconnecting the upper and lower support portions.

Knee braces of the type described serve to reduce pain in the knee joint, or to otherwise support the knee, by applying a three-point load to the leg. In arthritis braces, for example, a force is applied to the side of the knee opposite the affected compartment by causing a condyle pad attached to the knee brace to forcibly contact the side of the knee. This is typically accomplished by increasing the thickness of the adjacent condyle pad or by moving the pad closer to the knee using a screwdriver or other means. Alternatively, a force is sometimes applied opposite the affected compartment of the knee by means of an adjustable force strap which extends around the leg in a helical fashion from the upper support portion to the lower support portion. In either case, counteracting forces are applied to the leg in the medial/lateral plane above and below the knee on the side of the affected compartment by the upper and lower support portions. The resulting three-point load on the leg serves to reduce pain in the knee joint by reducing the load in the affected compartment of the knee.

Most of the knee braces of the type described above, however, are not custom fitted to a patient. A physician fits the knee brace, which is generally manufactured in generic sizes (e.g., small or medium), onto a patient based on the physician's judgment and patient's responses to the fitting. Currently available knee braces fail to provide any indication of the amount of force being applied by the brace to the knee joint to relieve the joint pain. When the clinician turns the jackscrew or a screwdriver to move the condyle pad towards or away from the knee, there is no feedback to the patient or the clinician of the amount of force the brace is applying to the knee joint. Each turn of the screw driver only results in arbitrary and subjective response from the patient regarding how comfortable he or she feels.

There is a need for a brace having features that provide feedback to the patient and clinician, to enable a clinician to better custom fit a brace to the patient, better relieve joint pain, and better track the progression of the patient's condition.

### BRIEF DESCRIPTION OF THE DRAWINGS

The following figures depict illustrative embodiments of the invention in which like reference numerals refer to like elements. These depicted embodiments may not be drawn to scale and are to be understood as illustrative of the invention and not as limiting.

FIG. 1 depicts a perspective view of a knee brace according to an illustrative embodiment of the invention.

FIG. 2 depicts a side view of a hinge assembly of the knee brace shown in FIG. 1.

FIG. 3 depicts an exploded view of the hinge assembly shown in FIG. 2.

FIG. 4 shows a force sensing resistor (FSR) according to an illustrative embodiment of the invention.

FIG. 5 shows an interior view of a hinge assembly shown in FIG. 3.

FIGS. 6-7 show an exemplary mating relationship between a force sensing resistor and a hinge unit according to an illustrative embodiment of the invention.

FIG. 8 shows an exemplary mating relationship between a force sensing resistor and a printed circuit board (PCB) according to an illustrative embodiment of the invention.

FIG. 9 shows an exemplary schematic of a circuit diagram for use in a PCB.

### DESCRIPTION OF EXEMPLARY EMBODIMENTS

A brace described herein includes a force sensor that measures the force applied by the brace onto the knee and a force indicator that displays the measured force. Light emitting diodes (LED) are disposed on the hinge assembly of the knee brace to indicate the amount of force the brace is applying to the knee to relieve the joint pain. In certain embodiments, when a specific force or force ranges are measured, different number of LEDs located on the hinge assembly light up depending on the measured force.

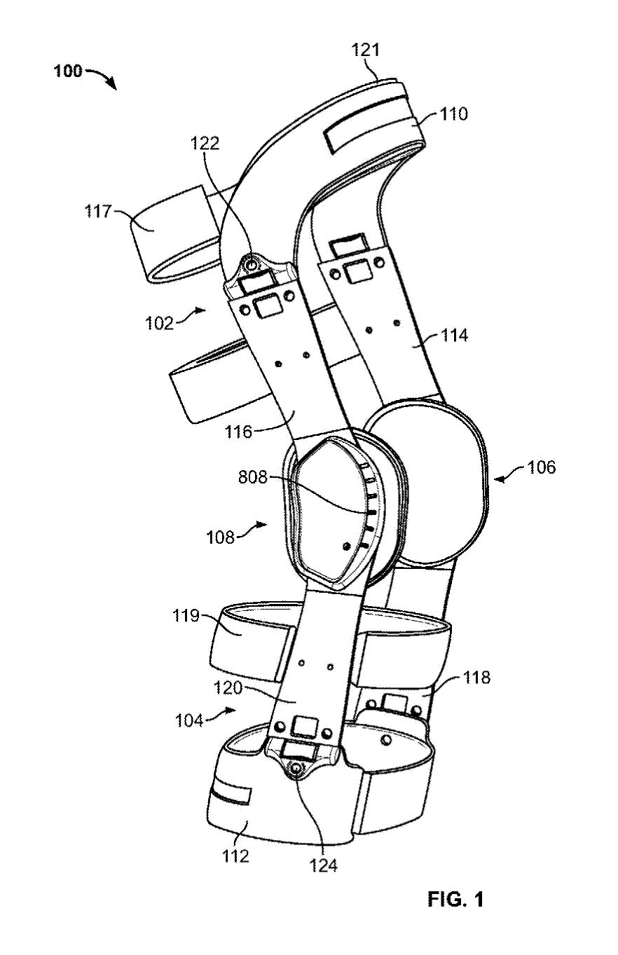

Turning to the illustrative embodiments, FIG. 1 shows a brace 100 including an upper support portion 102, a lower support portion 104, and medial and lateral hinge assemblies 106, 108. The upper support portion 102 includes an upper leg cuff 110 for positioning over the front of the thigh of the wearer and medial and lateral support arms 114, 116 extending from the upper leg cuff 110 to the medial and lateral hinge assemblies 106, 108. The lower support portion 104 includes a lower leg cuff 112 for positioning over the calf of the wearer and medial and lateral support arms 118, 120 extending from the lower leg cuff 112 to the medial and lateral hinge assemblies 106, 108. A first adjustment device 122 is located between the upper leg cuff 110 and the upper lateral support arm 116, and a second adjustment device 124 is located between the lower leg cuff 112 and the lower lateral support arm 120.

The upper support portion 102 is secured to the upper leg of the wearer with adjustable straps 117 which extend around the back of the upper leg. The lower support portion 104 is secured to the lower leg by adjustable straps 119 which extend around the front and back of the lower leg. For ease of adjustment, the straps 117, 119 are preferably fastened with a hook and pile fastener such as Velcro.

The upper and lower support portions 102, 104 are preferably made of aluminum, but may be made from any light-weight, high-strength metal, plastic, or composite material. The interior surface of each of the upper and lower support portions 102, 104 is covered with padding 121 to provide a comfortable fit against the leg of the wearer. The padding 121 is preferably made from a resilient foam material. However, inflated bladders, gels or other types of padding may also be used.

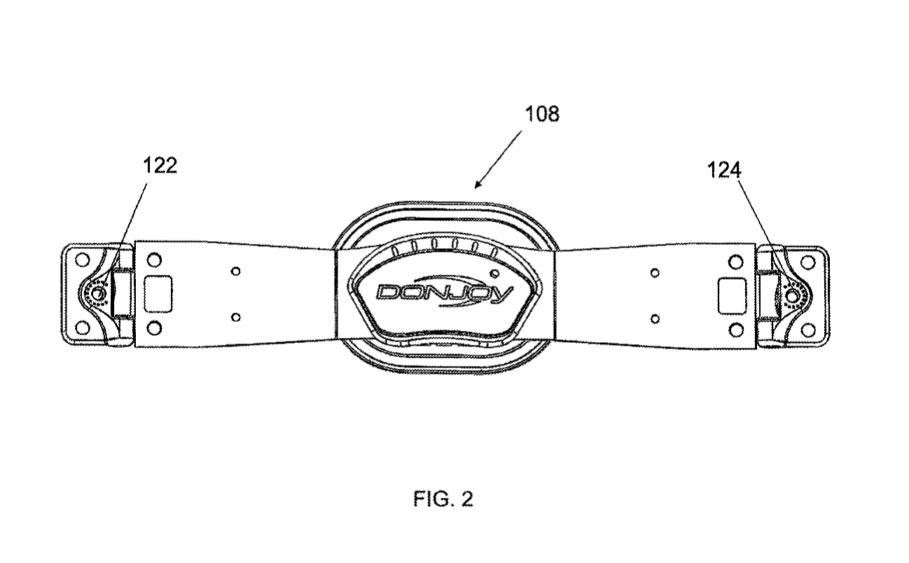

FIG. 2 shows the lateral hinge assembly 108 being positioned between the first and second adjustment devices 122 and 124. The lateral hinge assembly 108 includes a position adjusting mechanism (not shown) for applying force on the knee by moving a condyle pad attached to the lateral hinge assembly 108 towards the knee. The position adjustment mechanism is operatively connected to the first and second adjustment devices 122 and 124. An exemplary position adjusting mechanism is explained with reference to FIGS. 1-13 in U.S. Pat. No. 6,527,733 (Exhibit A), the teachings of which are incorporated herein by reference. In use, when a clinician turns an Allen-type wrench or a screwdriver clockwise within the adjustment device 122 and/or the adjustment device 124, the condyle pad positioned adjacent to the knee and attached to the lateral hinge assembly 108 moves closer to the knee, thereby applying force on the knee. When the clinician wishes to load the knee with more force, he or she turns the screwdriver more times in clockwise direction within the adjustment device. Turning the wrench counterclockwise moves the hinge assembly 108 away from the knee, which reduces the force applied to the knee. As noted above, the knee brace 100 includes force sensing and indicating features that measure and display the amount of force the brace is applying to the knee to relieve the joint pain. The brace 100 includes a plurality of LEDs that light up depending on the amount of force measured during the operation of the brace. The following figures provide greater detail on the force sensing and indicating features.

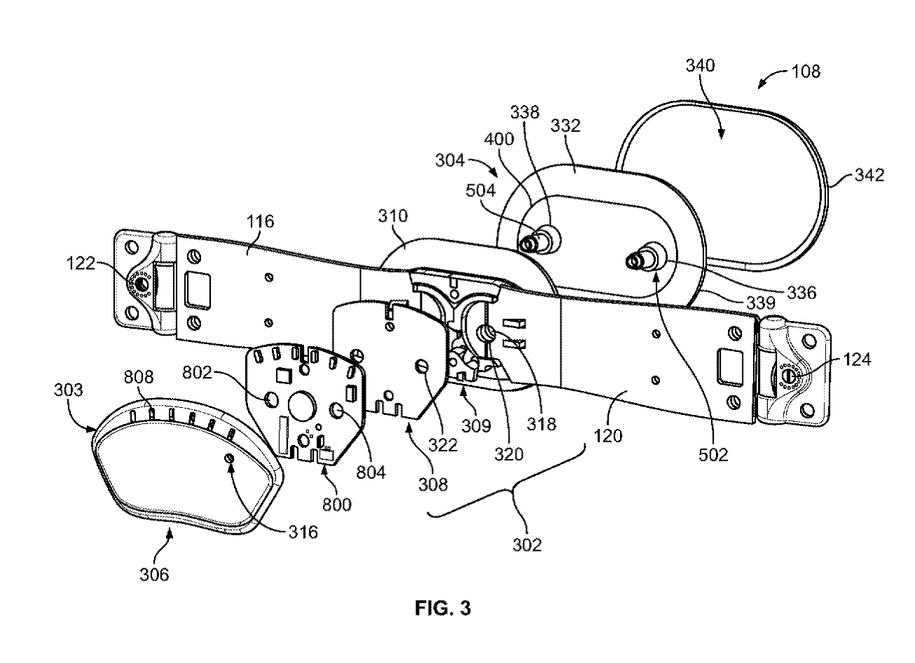

FIG. 3 is an exploded view of the lateral hinge assembly 108 of the knee brace 100. As shown, the lateral hinge assembly 108 includes a hinge unit 302, a sensing unit 304 disposed on the medial side of the hinge unit 302 for measuring a force applied by the brace onto a knee, and a display unit 306 that is operatively connected to the sensing unit 304 for displaying the force measured by the sensing unit 304. The hinge unit 302 also includes an outer hinge plate 308 and an inner hinge plate 310 enclosing a hinge mechanism 309. As shown, the display unit 306 and a printed circuit board (PCB) 800 are disposed on the lateral side of the hinge unit 302. The hinge unit 302, the PCB 800, and the display unit 306 are held in place by rivets 502 and 504. As shown, rivet 502 extends through each of holes 318, 320, 322, 804 of the inner plate 310, lateral support arm 120, the outer plate 308, and the PCB 800, respectively. Once secured, the display unit 306 is permanently disposed over the PCB 800 using, for example, adhesives, thereby securing the display unit 306 and the hinge unit 302 to the lateral arms 116 and 120 of the brace 100.

As shown, the display unit 306 includes a hinge cover 303 and a plurality of LED windows that light up in response to the amount of force measured by the sensing unit 304. The display unit 306 also includes an on/off switch 316 for activating and deactivating the sensing unit 304. In certain embodiments, the sensing unit 304 is activated only during the brace fitting sessions. In certain embodiments, the sensing unit 304 is turned on when a patient wishes to adjust the knee brace.

The sensing unit 304 includes a force sensing resistor (FSR) 400 and a layer of mating material (e.g., Velcro) 332 disposed on the lateral side of the force sensing resistor 400 for connecting the FSR 400 to the hinge unit 302. The layer 332 allows the FSR 400 to removeably attach to the hinge unit 302. The layer 332 also includes holes 336 and 338 for electrically connecting the FSR 400 to the PCB 800. The FSR 400 makes contact with the rivets 502 and 504 via the holes 336 and 338 of the layer 332 when the sensing unit 304 is engaged to the hinge unit 302 which is operatively connected to the PCB 800 via the rivets 502 and 504. In certain embodiments, the sensing unit 304 also includes a layer of adhesive material on the medial side of the FSR 400 for connecting the force sensing resistor 400 to a condyle pad 340. The condyle pad 340 makes contact with a patient's knee and is permanently engaged to the sensing unit 304. The condyle pad 340 is generally made from resilient foam or other cushioning material. Once the FSR 400 and the condyle pad 340 are engaged, the outer edges 339 and 342 of the FSR 400 and the condyle pad 340, respectively, are welded or laminated to create a unitary unit that is separately attachable to the hinge unit 302.

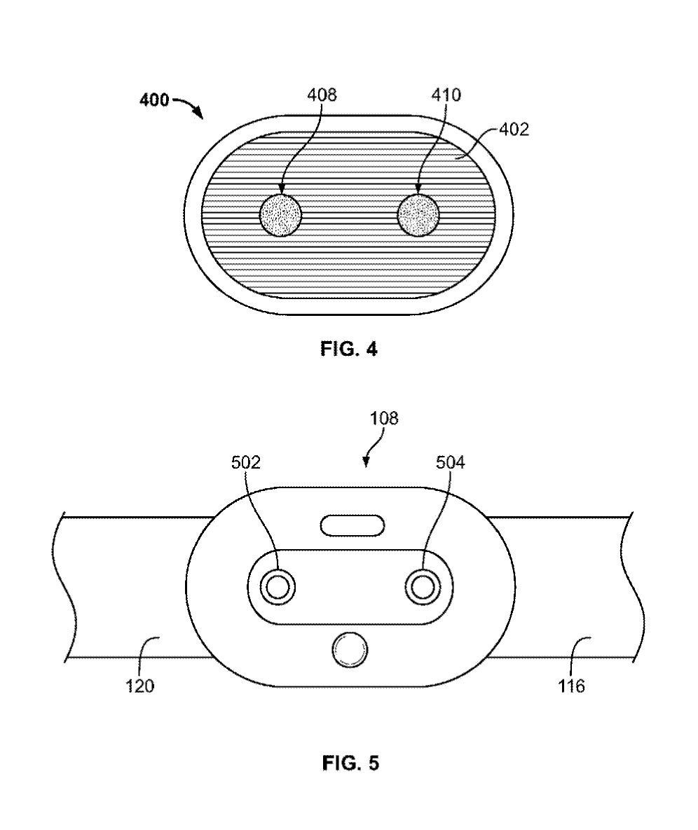

An exemplary mating relationship and the operation of the sensing unit 304 and the display unit 306 are described next with references to FIGS. 4-9. FIG. 4 shows an example of the FSR 400 including a force sensing film 402 having an electrical resistance R and +/− electrical terminals 408, 410. The force sensing film 402 is a piezoresistive conductive polymer and is created with two substrate layers and using polymer thick film ink screen printed on one of the substrates. It will be appreciated by those of skill in the art that the substrate can be any suitable material (i.e., polyester film) for use as a force sensing film. The ink formulation can be customized to provide the correct range of force sensitivity for the knee brace force sensing application. The other substrate layer is screen printed with interdigitated conductive electrode fingers. The two substrates are then positioned facing each other and adhered together with double-stick adhesive backed spacer material. Those of skill in the art will appreciate that the interdigiting conductive fingers can be created in various ways such as screen printing on a substrate with silver or silver/graphite conductive ink, or etched in copper and gold plated on a printed circuit board. The polymer thick film ink of the force sensing film 402 consists of both electrically conducting and non-conducting particles suspended in a matrix. Applying a force to the surface of the force sensing film 402 causes particles to touch the conducting electrode fingers, changing the resistance of the film. As force is applied to the force sensing film 402, the electrical resistance R decreases. The +/− electrical terminals 408, 410 comprise conductive contacts on the outside of the force sensing film 402. In operation, a current is passed through the FSR 400 by applying a voltage to the +/− terminals 408, 410. The more force applied to the FSR 400, the more conductive the force sensing film 402 becomes, and the more electrical current flows to the electrical terminals 408, 410.

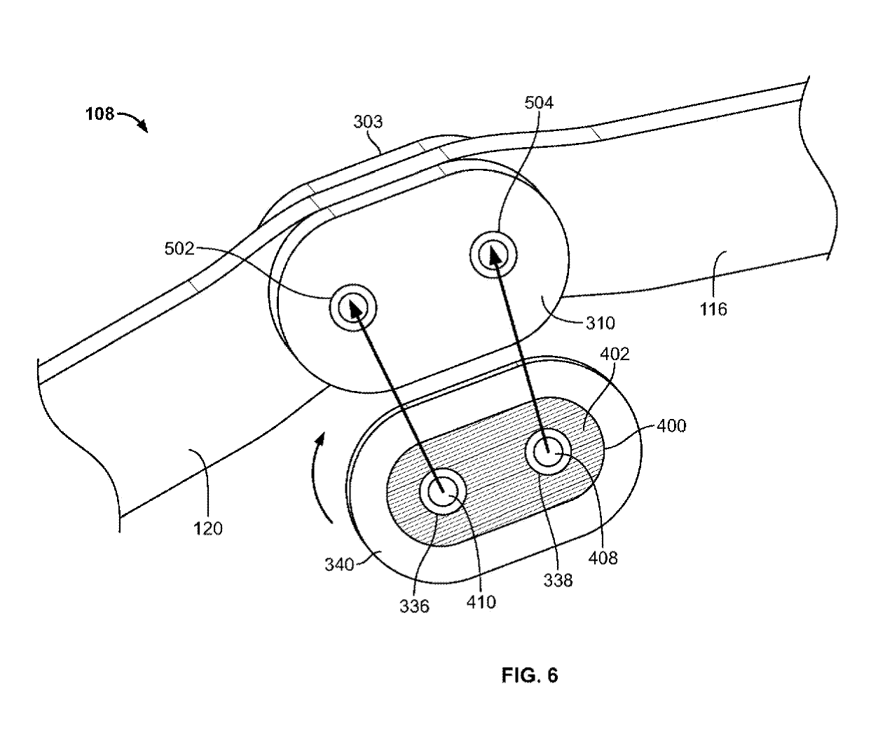

FIG. 5 shows an example of the rivets 502 and 504 positioned in the lateral hinge assembly 108 of the brace 100. As shown, the rivets 502 and 504 serve as both mechanical connectors for the hinge of the brace and as electrical conductors to conduct electrical signals between the force sensing resistor 400 and the PCB 800. Utilizing the hinge rivets as the electrical connectors between the FSR 400 and the PCB allows for an electrical connection without external wires.

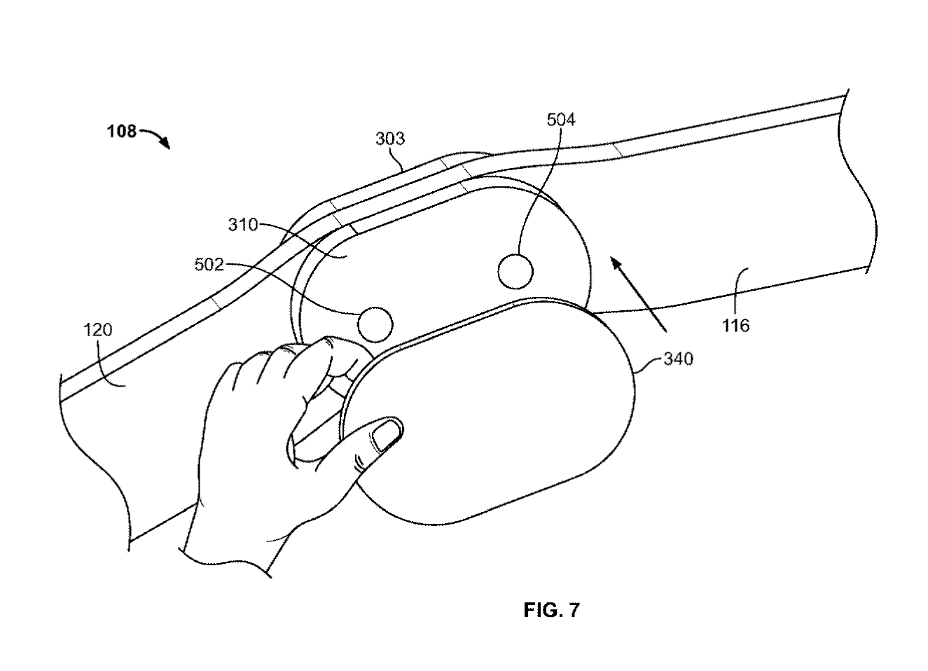

FIG. 6 shows an example of how the +/− electrical terminals 408, 410 of FSR 400 are electrically connected to the rivets 502 and 504. The +/− electrical terminals 408, 410 of FSR 400 are physically pressed up against or make contact with the metal rivets 502, 504. Because the rivets are made of metal, they are electrically conductive and will conduct a current passed from the +/− electrical terminals 408, 410 of FSR 400. FIGS. 6 and 7 also show that the FSR is attached to the condyle pad 340 of the brace. This allows for the FSR to be sandwiched between the condyle pad 340 and the hinge rivets 502, 504 so that a force applied by the patient's knee is effectively transferred through the condyle pad 340 to the FSR 400, wherein an electrical signal is created as a function of the force applied to the FSR 400. The electrical signal then passes through the +/− electrical terminals 408, 410 and to the rivets 502, 504 through direct physical contact between the electrical terminals 408, 410 and the rivets 502, 504. The rivets 502, 504 carry the electrical signal through the outer plate 308 to the PCB 800.

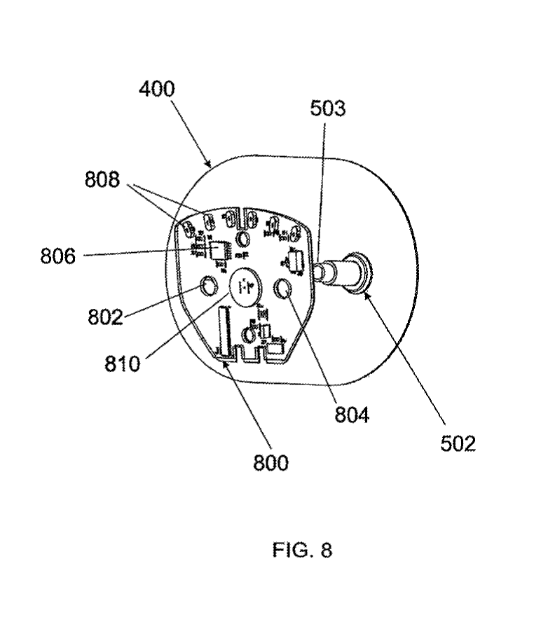

FIG. 8 shows an example of the connection between the FSR 400 and the PCB 800 thorough the rivets 502, 504. PCB 800 comprises through-holes 802, 804, microcontroller 806, LED's 808 and battery 810. It will be appreciated by those of skill in the art that PCB 800 may contain other circuit components in order to receive an electrical signal from FSR 400 and drive LED's 808. The rivets 502, 504 are mechanically and electrically connected to the PCB 800 via the through-holes 802, 804. For example, the tip 503 of rivet 502 passes through through-hole 804 and is crimped on the other side to securably connect the rivet 502 to the PCB 800 both mechanically and electrically. A similar process is used for the other rivet 504 (not shown) to connect to PCB 800 via through-hole 802. Electrical signals pass from the FSR 400, through the rivets 502, 504 and to the microcontroller 806 of the PCB 800. The microcontroller 806 may be programmed to drive LED's 808 in a specific pattern based on the amount of force applied to the FSR 400.

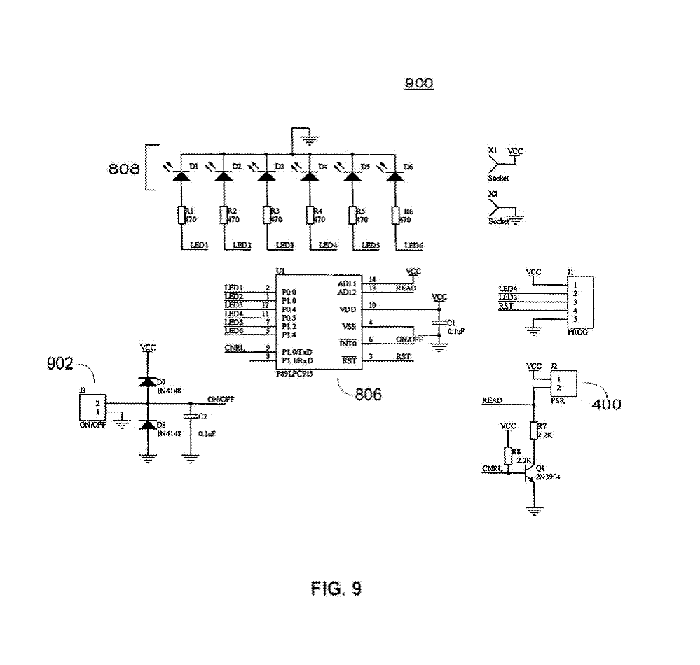

FIG. 9 shows an example circuit diagram 900 that could be used on PCB 800. The circuit diagram 900 comprises microcontroller 806, FSR 400, LED's 808 and on/off switch 902. It will be appreciated by those of skill in the art that the circuit diagram 900 may contain other circuit components for operation of the PCB 800. For example, other circuit elements can be used in order to receive an electrical signal from FSR 400 and to condition the electrical signal for input to microcontroller 806 and to drive LED's 808.

It will be appreciated by those of skill in the art that the medial hinge assembly may include the force sensing and indicating features as described herein.

What is claimed is:

1. An orthopedic brace having first and second support arms, the brace comprising:
a hinge unit having medial and lateral plates enclosing a hinge mechanism that joins the first and second support arms;
a sensing unit operatively connected to the hinge mechanism for sensing a force applied by the brace to a user's knee; and
a display unit that is operatively connected to the sensing unit for indicating a level of force applied to the knee.

2. The brace of claim 1, wherein the display unit includes a series of windows that light up incrementally in response to the force sensed by the sensing unit.

3. The brace of claim 1 comprising first and second adjustment devices for adjusting the position of the upper and leg lower cuffs of the hinge brace.

4. The brace of claim 1, wherein the sensing unit is disposed on the medial plate of the hinge unit and the display unit is disposed on the lateral plate of the hinge unit and is operatively connected to the sensing unit for displaying the force applied by the brace onto the patient's knee.

5. The brace of claim 1, wherein the sensing unit includes a force sensing resistor configured to change resistance with respect to the force applied by the user's knee.

6. The brace of claim 5, wherein the hinge unit comprises two electrically conducting rivets that pass through the hinge unit.

7. The brace of claim 6, wherein the force sensing resistor is disposed between a condyle pad and the rivets for transferring the force applied by the user's knee through the condyle pad to the force sensing resistor.

8. The brace of claim 7, wherein the force sensing resistor engages one end of the rivets, thereby creating an electrical connection to the rivets.

9. The brace of claim 8, wherein the opposite ends of the rivets are physically connected to a PCB creating an electrical connection thereto.

10. The brace of claim 8, wherein the force sensing resistor indicates to the PCB a signal indicative of a level of force being applied to the resistor upon tightening of the brace.

11. The brace of claim 5, wherein the force sensing resistor comprises a piezoresistive film that signals a tightening motion experienced by a brace support arm.

12. The brace of claim 7, wherein the force sensing resistor comprises +/− electrical terminals configured to make contact with the rivets, wherein the electrical terminals are configured to output an electrical signal that varies with respects to the force applied to the force sensing resistor.

13. The brace of claim 12, wherein the electrical signal is configured to pass through the +/− electrical terminals to the rivets.

14. The brace of claim 13, wherein the rivets carry the electrical signal through the lateral hinge plate to a PCB.

15. The brace of claim 12, comprising first and second adjustment devices for adjusting the position of the first and second support arms of the brace.

16. The brace of claim 15, wherein rotating the adjustment device in a first direction moves the condyle pad towards the knee, thereby applying force on the knee.

17. The brace of claim 16, wherein based on the electrical signal outputted by the electrical terminals, the PCB displays corresponding number of LEDs on the display unit.
